(19) United States
(12) Patent Application Publication
(10) Pub. No.: US 2021/0198608 A1
(43) Pub. Date: Jul. 1, 2021

(54) MICROFLUIDIC SYSTEMS FOR YEAST AGING ANALYSIS

(71) Applicant: UNIVERSITÉ DU LUXEMBOURG, Esch-sur-Alzette (LU)

(72) Inventors: Nicole PACZIA, Dusseldorf (DE); Paul JUNG, Strasbourg (FR); Pranjul SHAH, Ahmedabad (IN); Maria DIMAKI, Albertslund (DK); Winnie Edith SVENDSEN, Copenhagen (DK); Carole LINSTER, Esch-sur-Alzette (LU)

(21) Appl. No.: 17/271,482

(22) PCT Filed: Aug. 28, 2019
(86) PCT No.: PCT/EP2019/072988

§ 371 Date: Feb. 25, 2021

(30) Foreign Application Priority Data
Aug. 28, 2018 (LU) LU 100914

(51) Int. Cl. C12M 3/06; C12M 1/00; C12N 1/16

(52) U.S. Cl. CPC C12M 23/16; C12N 1/16; C12M 47/04

(57) ABSTRACT

The invention is directed to a microfluidic unit (101) for isolating and culturing yeast cells. This microfluidic unit (101) comprises a medium inlet, a medium outlet, a passage interconnecting the inlet and outlet, and in the passage a single cell trapping chamber (109). Moreover, this microfluidic unit (101) comprises further in the passage a daughter cells trapping chamber (111), this chamber (111) is placed downstream of the single cell trapping chamber (109), and configured for retaining the daughter cells while allowing offspring of these daughter cells to escape with a flow of the medium in the passage.

The work leading to this invention has received funding from the Fond National de Recherche (FNR) in Luxembourg under grant No. C16/BM/11339953.

### TECHNICAL FIELD

The invention relates to the field of cellular aging, and more particularly to the determination of the replicative lifespan of budding yeasts.

### BACKGROUND ART

In the last fifty years, the average of human life expectancy has increased rapidly. As people are living longer, the impact of age-related diseases takes in this context an important place. Advancing the understanding of the underlying molecular mechanisms of aging, as well their contributions to age-associated diseases, will have undoubtedly a profound impact on public health.

In aging studies, for the last few decades, the yeast has particularly emerged as a favourable model for understanding cell longevity and enabled significant contributions to the understanding of basic mechanisms of aging in eukaryotic cells. Besides the fact that this organism has a short generation time, and allows straightforward genetic approaches, the sequence similarities of its genome with mammalian cells makes it indeed effective to model human diseases.

As yeast is proliferating in an unsymmetrical way, there are two different approaches to determine its age, replicative lifespan (RLS) and chronological lifespan (CLS). RLS refers to the number of daughter cells produced by a mother cell before death and in some way resembles the aging of mammalian cells, such as fibroblasts and lymphocytes, which undergo a fixed number of divisions. In contrast, CLS is the time that a cell survives in non-dividing state, and this approach is used therefore as a model for the aging of non-prolifering cells as for instance neurons and cardiomycetes (Jung and al., 2015).

Both aging features have been investigated in the budding yeast Saccharomyces cerevisiae, and these studies permitted the discovery of widely conserved pathways involved in the regulation of lifespan from yeast to humans.

Concerning more particularly the determination of the RLS in yeast, the classical method consists to remove and count daughter cells from larger mother cells through manual dissection (Mortimer et al., 1959). This removal of daughter cells is necessary because a single mother cell becomes indiscernible from its exponentially dividing progeny after the daughter cell reached its final size. However, although this conventional method is conceptually simple, microdissection RLS assays are laborious, time-consuming, expensive and error prone.

As an alternative to this conventional microdissection technique, microfluidic technologies (Lee et al., 2012; Xie et al., 2012; Zhan et al., 2012; Fehrmann et al., 2013; Crane et al., 2014; Jo et al., 2015; Liu et al., 2015) have been recently developed these last years to study yeast aging, and have enabled to provide microfluidic platforms capable of tracking the whole lifespan of yeast cells. All currently known microfluidic platforms have similar working procedures. Firstly, young mother cells are immobilized in at least one microfluidic device. Then, the immobilized mother cells begin to grow and bud, producing daughter cells. When cytokinesis is completed, detached daughter cells are washed away automatically from their mother cells by a continuous flowing medium. By coupling the microfluidic device with time-lapse microscopy, microfluidic platforms obtained enable the tracking and the monitoring of trapped mother cells and thus the determination of their entire RLS.

This is the case, among others, in the published patent application US 2016/0281126 A1 which describes a microfluidic chip comprising 4 separate modules constituted of 4 microfluidic chambers in which 520 single cell-trapping structures allow to immobilize the same number of mother cells. The higher density of daughter cells is followed by time-lapse microscopy enabling the visualization and analysis of the complete RLS of single yeast cells.

Although such microfluidic platforms overcome low-throughput yeast RLS assays performed with the conventional microdissection technique, and provide also accurate analytical method at the single cell level, the fact remains that these microfluidic systems necessitate, to monitor continuously the multiple location of the single cell trapping structures, a continuous microscopy platform, either in a field of view or with a motorized platform. Beside the fact that such material is not trivial to set up, the huge amount of videos collected by these platforms and which need to be stored and analysed, requires specific facilities and material which can also be unaffordable for many research groups. Moreover, although these microfluidic platforms allow the tracking of individual yeast cells, they limit the tracking of generational phenotypic changes induced by genetic or environmental or chemical factor which can lead to discovery of new anti-aging drugs but also afford information to optimize parameter of yeast strains development to engineer new yeast strains.

### SUMMARY OF INVENTION

### Technical Problem

The invention has for technical problem to alleviate at least one of the drawbacks present in the prior art. More particularly, the invention has for technical problem to provide a high-throughput microfluidic platform for yeast RLS assays, which generates data faster than the other microfluidic systems, simple to use and implement, and moreover economical.

### Technical Solution

For this purpose, the invention is directed to a microfluidic unit for isolating and culturing yeast cells, comprising: a medium inlet, a medium outlet and a passage interconnecting said inlet and outlet; a single cell trapping chamber in the passage; wherein the microfluidic unit further comprises: a daughter cells trapping chamber in the passage, downstream of the single cell trapping chamber, and configured for retaining the daughter cells while allowing offspring of said cells to escape with a flow of the medium in said passage.

A mother cell trapping chamber is a first cell generation trapping chamber, and a daughter cells trapping chamber is a second cells generation trapping chamber.

According to a preferred embodiment, the daughter cells trapping chamber comprises a plurality of daughter cell sub-chambers and the passage comprises selective sub-passages each individually connecting one of said sub-chambers with the single cell trapping chamber.

According to a preferred embodiment, the selective sub-passages connect with the single cell trapping chamber at distinct locations arranged side-by-side.

According to a preferred embodiment, the selective sub-passages show a diameter or width greater than or equal to 2 μm and/or less than or equal to 5 μm.

According to a preferred embodiment, the passage comprises exit sub-passages each individually connecting one of said sub-chambers downstream with the medium outlet, each of said sub-passages showing a diameter greater than 5 μm.

According to a preferred embodiment, the selective sub-passages extend parallel to each other along a main direction of the unit, the exit sub-passages extending at least partially transversely to said main direction.

According to a preferred embodiment, the daughter cells trapping chamber forms a screen with openings, each opening being configured for retaining a daughter cell so that said chamber can trap a plurality of daughter cells.

According to a preferred embodiment, the openings of the daughter cells trapping chamber are more than 80, preferably more than 100.

According to a preferred embodiment, the openings of the daughter cells trapping chamber show, each, a diameter or width greater than or equal to 2 μm and/or less than or equal to 5 μm.

According to a preferred embodiment, the screen of the daughter cells trapping chamber extends along a main direction, the openings of said chamber being arranged in two parallel rows along said direction.

According to a preferred embodiment, the openings of the daughter cells trapping chamber are formed by pillars parallel to each other and extending between two substrates.

According to a preferred embodiment, the single cell trapping chamber shows an exit towards the daughter cells trapping chamber with a passage diameter or width greater than or equal to 2 μm and/or less than or equal to 5 μm.

According to a preferred embodiment, the passage between the single cell trapping chamber and the daughter cells trapping chamber forms at least one, preferably several meanders.

According to a preferred embodiment, the single cell trapping chamber is a mother cell trapping chamber, said unit further comprising a grand-mother cell trapping chamber in the passage, upstream of said mother cell trapping chamber.

According to a preferred embodiment, the passage comprises at least a drain by-pass fluidly connecting the grand-mother cell trapping chamber to the medium outlet.

According to a preferred embodiment, at least a drain by-pass shows a diameter that is greater than 5 μm.

According to a preferred embodiment, the passage between the grand-mother cell trapping chamber and the mother cell trapping chamber shows a diameter or width that is greater than or equal to 2 μm and/or less than or equal to 5 μm.

The invention is also directed to a microfluidic device comprising: an inoculation channel with a fluid inlet; a waste channel with a fluid outlet; and microfluidic units arranged side-by-side each with the medium inlet connected to the inoculation channel and the medium outlet connected to the waste channel; wherein each of the microfluidic unit is according to the invention, and the set of inoculation channels are connected to form the unique inoculation channel, and the set of waste channels are connected to form the unique waste channel.

According to a preferred embodiment, the microfluidic device further comprises a rotatable basis supporting the inoculation channel, the waste channel and the microfluidic units, said units having each a main direction extending along a radius of said basis.

The invention is also directed to a platform comprising a liquid handling set up, a computer, a microscope with a camera and at least a microfluidic device; wherein the at least a microfluidic device is according to the invention.

According to a preferred embodiment, the platform further comprises a centrifuge and the at least a microfluidic device is according to the invention.

The invention is also directed to a method of isolating and culturing a plurality of yeast cells comprising the following steps: providing a platform for yeast-aging analysis comprising a liquid handling set up or a centrifuge, a computer, a microscope with a camera, and at least one microfluidic device; injecting suspended yeast cells through the inoculation channel; trapping in each daughter cells trapping chambers the entire progeny of each mother cells captured in each single cell trapping chamber, taking photos of the daughter cells tapping chambers, wherein the at least one microfluidic device is according to the invention.

### Advantages of the Invention

This invention is particularly interesting in that the configuration of microfluidic units allows to screen the entire offspring/progeny of a single mother cell, namely in this case all daughter cells of this single cell; These microfluidic units are particularly interesting in that being incorporated in microfluidic devices of platforms according to the invention, they eradicate the need for online monitoring of the microfluidic devices. Indeed, the outcome of the assays, namely the daughter cells of each virgin mother cell, retained in the daughter cells trapping chambers, can be easily determined with a single image taken at the end of the assays. This invention is all the more interesting in that microfluidic devices according to the invention can be designed also as a stand-alone centrifugal device which can be operated by using commercial bench centrifuges, and thus allows to avoid the use of a microfluid setup. This invention is interesting in that it allows to provide high-throughput microfluidic platforms for yeast RLS assays, generating more rapidly and easily accurate data than a manual RLS determination or a high throughput video-based microfluidic determination since compared to the first technique, an operator does not need to be always present and assays do not need to be interrupted overnight, and compared to the second technique the parallelism of a high number of independent experiments can be easily realised by taking a number of limited photos, avoiding therefore the constraint of the movement of the video-microscope, the treatment and the storage of a huge amount of videos.

### BRIEF DESCRIPTION OF THE DRAWINGS

FIG. 1 is a schematic view of the upper face of a first embodiment of a microfluidic unit according to the invention.

FIG. 2 is a schematic view of the upper face of a second embodiment of a microfluidic unit according to the invention.

FIG. 3 is a schematic view of a first and preferred alternative of the second embodiment of the microfluidic unit illustrated FIG. 2.

FIG. 4 is a partial and enlarged view of the first and preferred alternative represented in FIG. 3.

FIG. 5 is schematic view of a second alternative of the second embodiment of the microfluidic unit illustrated in FIG. 2.

FIG. 6 is schematic view of a third alternative of the second embodiment of the microfluidic unit illustrated in FIG. 2.

FIG. 7 is a partial schematic view of a microfluidic device according to a first embodiment of the invention.

FIG. 8 is a partial and schematic view of a microfluidic device according to a second embodiment of the invention.

FIG. 9 illustrates a method for the determination of the RLS of yeast cells according to the invention.

### DESCRIPTION OF AN EMBODIMENT

FIG. 1 illustrates a schematic view of the upper face of a microfluidic unit according to a first embodiment of the invention. This microfluidic unit 1 is dedicated for the isolation and culturing of yeast cells, in particular budding yeast cells, and is crossed by a longitudinal passage 7 connected at one extremity to a medium inlet 3 and at the other extremity to a medium outlet 5 through which flows a medium in which a suspension of yeast cells has been previously inoculated. In line with the two inlets 3; 5, the passage 7 of the microfluidic unit 1 comprises successively a single cell trapping chamber 9 and a daughter cells trapping chamber 11. The single cell chamber 9 is actually a single mother cell chamber 9 and is placed upstream to the daughter cells trapping chamber 11. This last chamber 11 is configured for retaining the daughter cells of the single mother cell which has been captured in the single cell trapping chamber 9, while allowing the progeny of these daughter cells to escape with a flow of the medium through the medium outlet 5.

In this first embodiment of the microfluidic unit 1 according to the invention, the daughter cells trapping chamber 11 is formed by a plurality of daughter cell sub-chambers 13 and the passage 7 comprises further selective sub-passages 8 and exit sub-passages 12.

Each selective sub-passage 8 presents a diameter or width generally greater than or equal to 2 μm and/or less than or equal to 5 μm and is individually connected to one of said sub-chambers 13 with the single cell trapping chamber 9, the connection of each sub-chamber 13 with the single cell trapping chamber 9 being at distinct locations arranged side-by-side. In addition, these selective sub-passages 8 extend parallel to each other along a main direction of the microfluidic unit 1. This main direction is horizontal and in line with the medium inlet 3, the single cell trapping chamber 9, the daughter cells trapping chamber 11 and the medium outlet 5.

Concerning the exit sub-passages 12, they have a diameter or width generally greater than 5 μm, and each are individually connected to one of said sub-chambers 13 downstream with the medium outlet 5. Relative to the selective sub-passages 8, the exit sub-passages 12 extend in turn at least partially transversely to the main direction followed by the selective sub-chambers 8.

Preferably, and as it can be seen in FIG. 1, the first embodiment of the microfluidic unit 1 further comprises in the passage 7, between the medium inlet 3 and the medium outlet 5, a grand-mother cell trapping chamber 6 which is placed upstream and in line with the mother cell trapping chamber 9 and connected to the medium inlet 3 by a short channel of 10 μm of diameter or width. The grand-mother cell trapping chamber 6 comprises also at least a drain by-pass 10 fluidly connected to the medium outlet 5. The inlet of each drain by-pass 10 has a diameter or width greater than or equal to 4 μm and/or less than or equal to 7 μm. On the FIG. 1, in this case, the microfluidic unit 1 shows only one drain by-pass 10.

It has to be also observed in FIG. 1 that advantageously the passage between the grand-mother cell trapping chamber 6 and the mother cell trapping chamber 9 forms a short constriction which is characterized by a diameter or width that is greater than or equal to 2 μm and/or less than or equal to 5 μm. The grand-mother cell trapping chamber 6 is actually dedicated to capture a single cell, usually called grand-mother cell, from the suspension of yeast cells which circulate through the medium inlet 3. The grand-mother cell retained, has in fact no determined age but is still enable to bud. The first cell produced by the grand-mother cell retained in the grand-mother cell trapping chamber 6 is directed through the short constriction/passage to the mother cell trapping chamber 9 to form a virgin mother cell of which the entire offspring/progeny (i.e. all its daughter cells) is then captured in the subsequent daughter cell sub-chambers 13. The at least drain by-pass 10 connected to the grand-mother cell trapping chamber 6 serves to evacuate by flow to the medium outlet 5 all additional offspring of the grand-mother cell. In the same way, the exit sub-passages 12 serve to eliminate the progeny through the medium outlet 5 of each daughter cell (of the mother cell) which have been trapped in the daughter cells sub-chambers 13. The geometry of the daughter cell sub-chambers 13 of the first embodiment of the microfluidic unit according to the invention is such that only one daughter cell of the mother cell, captured in the mother cell trapping chamber 9, enters in the first daughter cell sub-chamber 13 while the next daughter cell of the same mother cell is directed to the next free daughter cell sub-chamber 13 by increasing hydrodynamic resistance, same for the other next daughter cells.

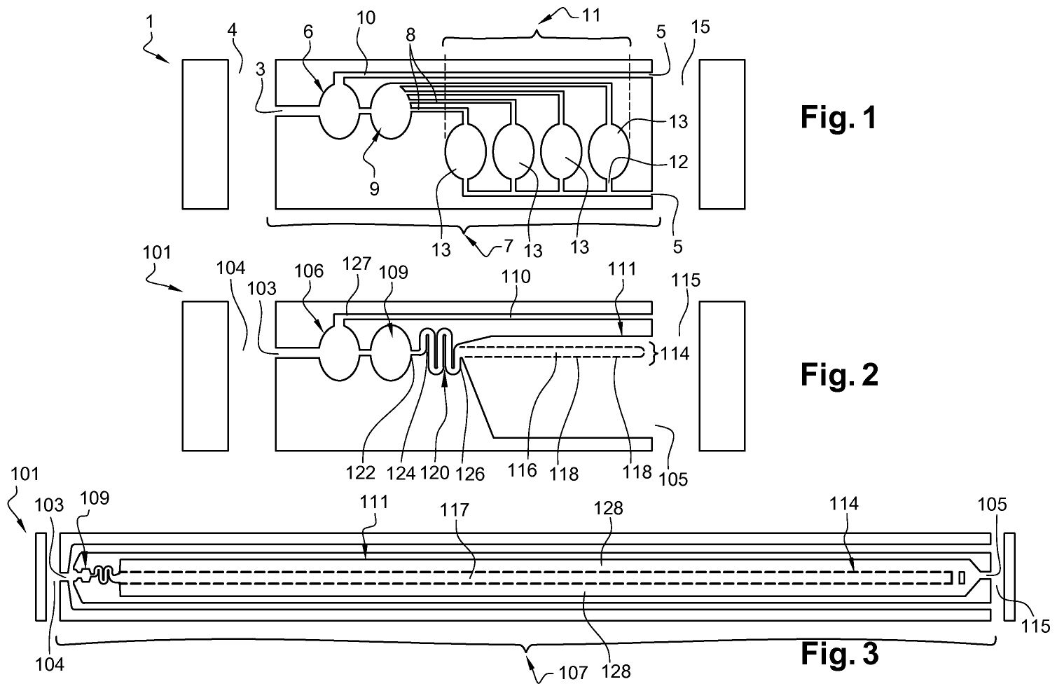

FIG. 2 is a schematic view of a second embodiment of a microfluidic unit according to the invention. In this microfluidic unit 101, the daughter cells trapping chamber 111 forms a screen 114 with openings 116 so that this chamber 111 can trap a plurality of daughter cells. The openings 116 of the screen 114 are more than 80, preferably more than 100 and show, each, a width greater than or equal to 2 μm and/or less than or equal to 5 μm. These openings 116 are in fact formed by pillars 118 parallel to each other and extending between two rows, and they constitute escape channels 116 for the progeny of each of the captured daughter cells in the daughter cells trapping chamber 111.

In this second embodiment, it has to be also noted that the daughter cells trapping chamber 111 extends along a main direction, which is a horizontal direction in line with the single cell trapping chamber 109 and the medium outlet 105, and that similarly, the two rows of openings/escape channels 116 are arranged along the same main horizontal direction, forming thus a main horizontal channel 117 with an uniform diameter greater than or equal to 4 μm and/or less than or equal 7 μm. In parallel, the pillars 118 of the daughter cells trapping chamber 111, which delimit each escape channel 116 of the main horizontal channel 117, are square, rectangular, oval or round-shaped, and prevent the daughter cells which are trapped at the end of the main horizontal channel 117, from being flushed into the medium outlet 105.

Generally, the main horizontal channel 117 of the daughter cells trapping chamber 111 is in fact long enough to allow 100 daughter cells to be trapped, that makes approximatively a length of about 500 μm.

It should be also observed that in this second embodiment of the microfluidic unit according to the invention, the single cell trapping chamber 109 has an exit 122 towards the daughter cells trapping chamber 111 with a passage diameter or width greater than or equal to 2 μm and/or less than or equal to 5 μm. This said passage forms, as it is the case in FIG. 2, at least one meander 120, preferably several meanders 120, which presents an inlet 124 and an outlet 126. The meander 120 connects the single cell trapping chamber 109 to the horizontal main channel 117 of the daughter cells trapping chamber 111. Its diameter or width is comprised between 2-5 μm at its inlet 124 and between 4-7 μm at its outlet 126. The length of the meander 120 allows in fact the daughter cells of the single cell trapping chamber 109 to grow in size, before they reach the main horizontal channel 117. It should be also specified that the inlet 124 of the meander 120 is identical to the size of the outlet 122 of the single cell trapping chamber 109, and the outlet 126 of the meander is identical to the diameter or width of the main horizontal channel 117.

As for the first embodiment of the microfluidic unit 1 of the invention, the single cell mother trapping chamber 109 of the second embodiment of the microfluidic unit 101 is actually a mother cell trapping chamber 109. Moreover, and also preferably this microfluidic unit 101 further comprises in the passage 107, between the medium inlet 103 and the medium outlet 105, a grand-mother cell trapping chamber 106 which is placed upstream and in line with the mother cell trapping chamber 109 and connected by a short channel of 10 μm diameter to the medium inlet 103. The grand-mother cell trapping chamber 106 comprises also at least a drain by-pass 110 fluidly connected to the medium outlet 105. Although in FIG. 2, the microfluidic unit 101 shows only one drain by-pass, preferably it comprises two drain by-passes 110 (see FIG. 3), each drain by-pass 110 presenting an inlet 127 having a diameter or width greater than or equal to 4 μm and/or less than or equal to 7 μm.

In the microfluidic unit 101 of the second embodiment of the microfluidic unit according to the invention, the passage between the grand-mother cell trapping chamber 106 and the mother cell trapping chamber 109 is also characterized by a diameter or width that is greater than or equal to 2 μm and/or less than or equal to 5 μm.

Similarly to the microfluidic unit 1 of the first embodiment, the grand-mother cell chamber 106 of the microfluidic unit 101 of the second embodiment is dedicated to capture a single grand-mother cell, from the suspension of yeast cells which circulate through the medium inlet 103. The first cell produced by the grand-mother cell retained in the grand-mother cell trapping chamber 106, flows then to the mother cell trapping chamber 109 to form a virgin mother cell. The entire progeny of this last cell is then captured in the main horizontal channel 117. The drain by-passes 110 connected to the grand-mother cell trapping chamber serve in fact to evacuate by flow to the medium outlet 105 the rest of the progeny of the grand-mother cell. In the same way the escape channels/openings 116, serve to eliminate through the medium outlet 105 the progeny of each daughter cell trapped in the main horizontal channel 117 of the daughter cells trapping chamber 111.

It should be also noticed that the first and the second embodiments of the microfluidic unit according to the invention preferably comprise in addition an inoculation channel 4; 104 and a waste channel 15; 115, the inoculation channel 4; 104 being connected upstream of the medium inlet 3; 103, and the waste channel 15; 115 being connected downstream to the medium outlet 5; 105 (see FIGS. 1 and 2). In general, these two channels 4; 104/5; 105 belong to the passage 7; 107 of the microfluidic unit 1; 101 which is comprised between the medium inlet 3; 103 and the medium outlet 5; 105 and defined above, or can be also independently formed, and connected to the medium inlet 3; 103 and the medium outlet 5; 105, respectively.

FIG. 3 is a schematic view of a first and preferred alternative of the second embodiment of the microfluidic unit 101 of the FIG. 2. In this alternative, the screen 114 with openings 116 formed by the daughter cells trapping chamber 111 is in the centre of this chamber, and delimits two side channels 128 which extend along the main horizontal direction above and below the main horizontal channel 117. These side channels 128 have a diameter or width greater than or equal to 4 μm and/or less than or equal to 7 μm, and enable to evacuate by flow the offspring/progeny of each daughter cell retained in the daughter cells trapping chamber 111 through the escape channels 116 towards the medium outlet 105. In this alternative, the inoculation channel 104 and the waste channel 115 belong both to the horizontal passage 107 of the microfluidic unit 101. Advantageously, these channels 104, 115 also present a transversal form of 20 μm and cross in larger the microfluidic unit 101.

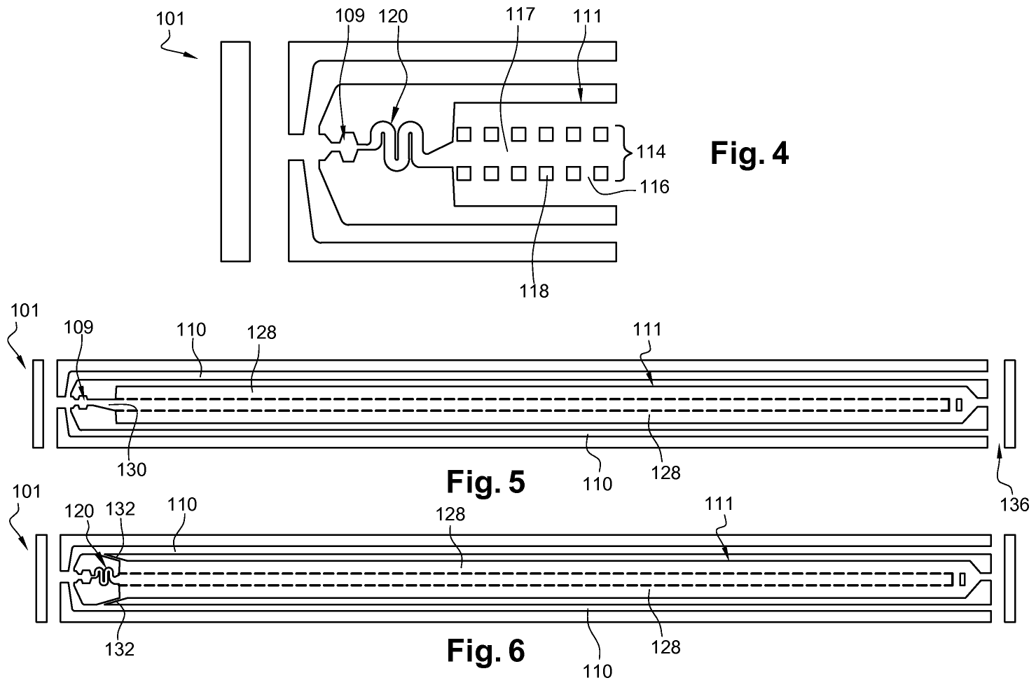

FIG. 4 is a partial and enlarged view of the first and preferred alternative of the second embodiment of the microfluidic unit 101 illustrated in FIG. 3. This FIG. 4 allows to observe that the size of the meander 120, present between the single cell trapping chamber 109 and the daughter cells trapping chamber 111, slightly increases from its inlet 124 to its outlet 126. In this way, the daughter cells, flowing from the mother cell trapping chamber 109, have time to get mature and grow sufficiently, for not having the possibility to escape through the escape channels 116 of the screen 114, once they have penetrated in the daughter cells trapping chamber 111.

FIG. 5 is a schematic view of a second alternative of the second embodiment of the microfluidic unit of the FIG. 2. In this alternative, and as in this case in the present FIG. 5, the microfluidic unit 101 presents, instead of a meander 120, a horizontal channel 130 the size of which increases between the single cell trapping chamber 109 and the daughter cells trapping chamber 111.

FIG. 6 is schematic view of a third alternative of the second embodiment of the microfluidic unit of the FIG. 2. As for the first alternative (see FIGS. 3 and 4) this alternative comprises a meander 120 but also additional connexions 132 positionned between at least one drain by-pass 110 and a side channel 128 of the daughter cells trapping chamber 111. These additional connexions 132 form in fact passages which serve to increase the flow in the side channels 128.

Advantageously, in each alternative of the second embodiment of the microfluidic unit, the outlet of each single cell trapping chamber 109 can comprise a vertical structure of 2 μm. As the inlet and the outlet of this chamber 109 have the same size, this vertical structure can limit the exit of the mother cell captured.

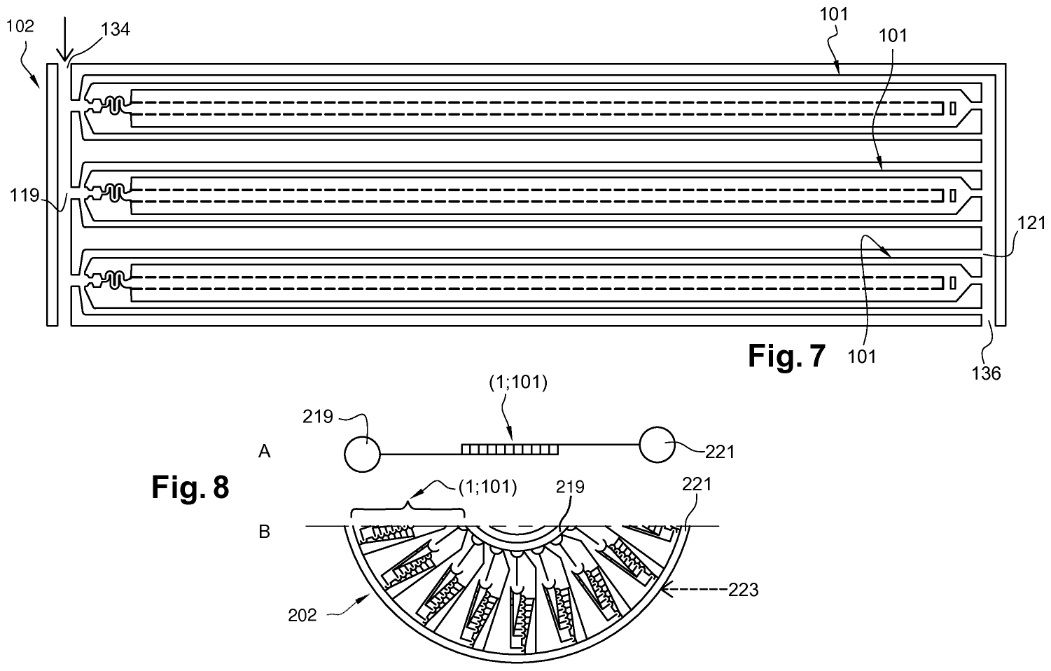

FIG. 7 is a partial schematic view of the upper face of a first embodiment of a microfluidic device according to the invention. Preferably, this microfluidic device 102 is formed by at least 100 microfluidic units 1, 101. These set of microfluidic units 1; 101 are formed according to the first embodiment or the second embodiment of the invention.

Moreover, in this microfluidic device 102, the inoculation channels 4; 104 of each microfluidic unit 1; 101 are connected to a unique inoculation channel 119, and all the waste channels 5; 105 are connected to a unique waste channel 121, the inoculation channel 119 being connected to a fluid inlet 134, and the inoculation channel 121 being connected to a fluid outlet 136.

On the FIG. 7, in this case, only three microfluidic units 101 are represented on the microfluidic device 102. These microfluidic units 101 are constructed according to the first and preferred alternative of the second embodiment of the invention. Each microfluidic unit 101 having preferably a length of about 747 μm and a width of 58 μm.

FIG. 8 is a partial and schematic view of a second embodiment of a microfluidic device according to the invention. This microfluidic device 202 is in fact formed with microfluidic units 1; 101 according to the first embodiment or either to the second embodiment of the invention, but in this embodiment these microfluidic units 1; 101 have a main direction extending along a radius of a rotable basis 223. As in the microfluidic device 102, the second microfluidic device 202 and the inoculation channel 4; 104 of each microfluidic unit 1; 101 are also connected to a unique inoculation chamber 219, and all the waste channels 5; 105 are connected to a unique waste channel 221. The rotable basis 223 (not visible on the FIG. 8 since positioned below) supports the inoculation channel 219, the waste channel 221 and the microfluidic units 1; 101. In this FIG. 8, (A) shows particularly a single microfluidic unit 1; 101 and (B) presents a part of the upper face of the microfluidic device 202.

Compared to the microfluidic device 102 the microfluidic device 202 can also be not connected to a fluid set-up, and it is based on centrifugal microfluidics technology which allows utilisation of any commercial available centrifuges.

Advantageously, in this invention at least a microfluidic devices 2; 102 according to the invention and presented in FIGS. 7 and 8, can be installed in a platform for RLS analysis of yeast cells. This platform is further connected to a microscope with a camera, a computer, and a liquid handling set up. Platforms having at least a microfluidic device according to the second embodiment use a centrifuge.

The material of the microfluidic devices 2; 102 according to the invention, and consequently of each microfluidic unit 1; 101, is polydimethylsiloxane (PDMS), which is a transparent in visible/UV ranges and gas-vapour-permeable elastomer. The microfluidic units 1; 101 are generally formed by two superposed planar substrates of PDMS.

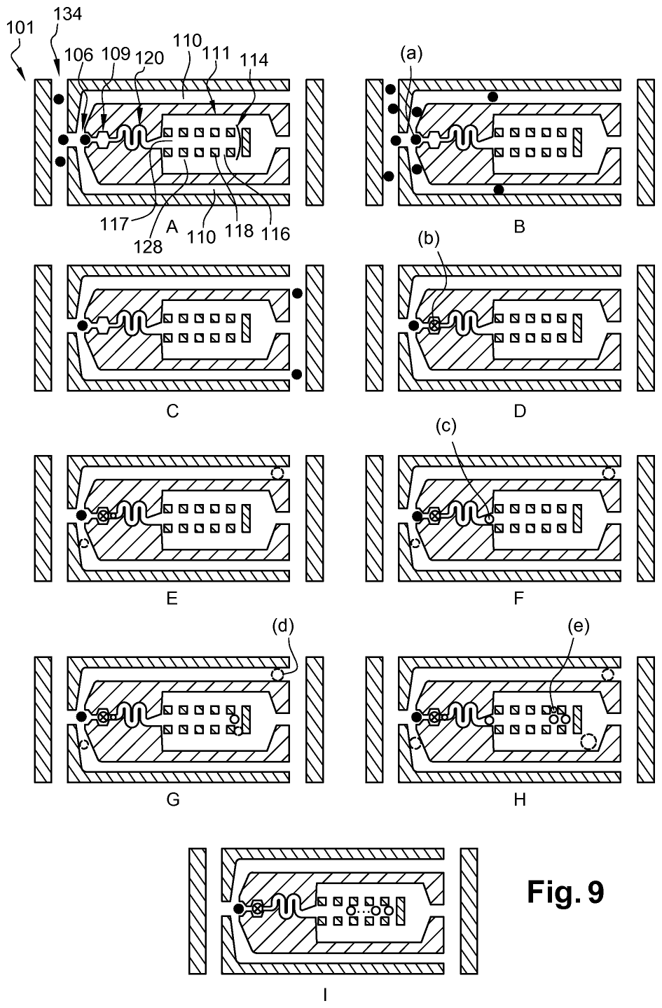

FIG. 9 illustrates particularly a method for the determination of the RLS yeast cells according to the invention. In this case the FIG. 9 shows the second and the third step of this method. This method is illustrated in particular with the microfluidic device 102 which is formed with microfluidic units 101 belonging to the preferred alternative of the second embodiment of the invention and which comprises grand-mother cell trapping chambers. In order to best describe these two steps of the method, separate blocks numerated A to I are illustrated.

(A) A syringe filled with diluted budding yeast cells, such for instance Saccharomyces cerevisiae, is connected to the fluid inlet of a microfluidic device 102 of a platform comprising a microscope with a camera, and a microfluidic set up. Media with cells are thus flushed through the inoculation channel 119 and by applying a selective microfluidic pressure at the inlet of each grand-mother cell trapping chamber 106, which have a not restricted size of 10μm, this enables (by chance) a single cell to enter in each of these chamber 106.

(B) (C) As soon as these single cells, commonly designed grand-mother cells (a), are each captured in a grand-mother cell trapping chamber 106, the syringe used for the inoculation is changed by a syringe with free medium to remove the remaining cells (d) from the inoculation channel.

(D) The positioning of the outlet of each grand-mother cell trapping chamber 106 with the inlet of the corresponding mother cell tapping chamber 109, enables the first (progeny) cell (b) of a grand-mother cell (a) to leave the grand-mother cell trapping chamber 106 to the corresponding mother cell trapping chamber 109. The 3 μm outlet of each grand-mother cell trapping chamber 106, which is connected directly to the 3 μm inlet of a mother cell trapping chamber 109, prevents the entrance of all additional progeny cell of the grand-mother cells (a) into the mother cell trapping chambers 109. The additional progenies of the grand-mother cells (d) are evacuated in parallel by the drain channels 110 to the medium outlet and the waste channel 121.

(D) Each cell (b) captured in each mother cell trapping chamber 109 grows in size, the outlet of 3 μm of these chambers 109 as well as the meander 120 preventing them from getting out.

(E) The following step consists of the entrance of the first daughter cell (c) of each mother cell (b) in the meanders 120 of the microfluidic units 101, in which they grow in size.

(F) These first daughter cells (c) are then flushed and captured in the end of the main horizontal channel 117 of each daughter cell trapping chambers 111, while their progenies are eliminated through the openings or escape channels 116 to the medium outlet and the waste channel 121, via the side channels 128 of the microfluidic units 101.

(H) At the same time, each second daughter cell (c) of each mother cell (b), in its turn, enters in the meander 120, grow in size and is flushed also, as the first daughter cell (c), into the end of the main horizontal channel 117 of a daughter cell trapping chamber 111.

(I) The same process occurs for each third daughter cell (c) of each mother cell (b), and therefore for all the progenies of the daughter cells (c).

Finally, each daughter cell trapping chambers 111 of the microfluidic device 102 contained all the progenies (c) of a mother cell (b). Once the cells (c) are captured they are aligned inside the main horizontal channel 117 of the daughter cells trapping chambers 111 and a photo can be taken and the cells counte to determine the RLS of a yeast strain.

The advantages of the microfluidic devices 102; 202 according to the invention are to provide an accurate, easier, faster and economic determination of the RLS of the yeast cells, than the conventional microdissection and the known high throughput microfluidic devices using time-lapse microscopy. Moreover, with the second embodiment of the microfluidic device 202, it is possible to perform RLS assays on any commercial centrifuge, avoiding thus the need to use a fluid set up and a pump. As the results of the counting in the microfluidic devices 102; 202 are easy and fast to obtain, since at least a photo has to be taken, the RLS assays can be repeated several times, permitting to get accurate and high throughput data.

In general, this invention has the advantage of a more accurate and fast determination of the lifespan of a single yeast cell (Saccharomyces cerevisiae), thus making easier the study of the molecular mechanisms of aging in eukaryotes cells. Indeed, the microfluidic devices with platforms, according to the invention, provide a simplified, automated, stand-alone, high throughput and integrated system which removes the time and price barrier for yeast lifespan determination. This invention is even more interesting in that it can be also used in different research areas employing yeast as a research organism, but also in different markets such as for instance: brewery, pharmaceutical, frozen bakery and bioremediation.
concerning the brewery, the brewing process depends highly on yeasts as natural factor. The present invention can offer therefore decisive advantages to perform large screening/phenotyping of yeast cells for identifying suitable yeast strains which are essential for successful high-gravity brewing.
in the pharmaceutical market, yeast cells are also used as model for drug screening approaches, thereby microfluidic devices and platforms according to the invention can be very useful tools to perform easily screenings of drugs or large collections of compounds.
with respect to frozen bakery, frozen products and especially bread, strongly depend on the stability of yeasts in the frozen dough during the storage. To prolong product shelf life in frozen status, yeast strains with high cryoresistance are needed. This invention can offer the possibility to identify ideal age for yeast usage in frozen products and/or to identify more suitable yeast strains by characterising age-related effects on products linked to yeast cryoresistance, such as trehalose and protein production.

In the bioremediation field, the approach of yeast-based bioaugmentation is used for soil and water purification of contaminated sites containing heavy metals and/or organic polluants. The microfluidic devices and the platforms according to the invention can offer the possibility to separate and screen yeasts from site-specific heavy-metal polluted river and lakes, for tolerance on high heavy pollutions under different environmental conditions in order to establish new strains collections.

### REFERENCES

Jung P. P., Christian N., Kay D. P., Skupin A., Linster C. L.; Plos one. 2015, 1-20.
Mortimer R. K., Johnston J. R.; Life span of individual yeast cells. Nature 1959; 183:1751-1752.
Xie Z. W., Zhang Y., Zou K., Brandman O., Luo C.X., Ouyang Q. Li H.; Molecular phenotyping of aging in single yeast cells using a novel microfluidic device: Molecular phenotyping of aging. Aging Cell. 2012; 11:599-606.
Zhang Y., Luo C. X., Zou K., Xie Z. W., Brandman O., Ouyang Q., Li H.; Plos One. 2012, 7.
Lee S., Avalos Vizcarra I., Huberts D. H. E. W., Lee L. P., Heinemann M.; Proc. Natl. Acad. Sci. USA. 2012, 109, 4916.
Fehermann S., Paoletti C., Goulev Y., Ungureanu A., Aguilaniu H., Charvin G; Cell Rep. 2013, 5, 1589.
Crane M. M., Clark I. B., Bakker E., Smith S., Swain P. S.; Plos one. 2014, 9 e100042.
Jo M. C., Liu W., Gu L., Dang W. W., Qin L. D.; Proc. Natl. Acad. Sci. USA. 2015, 112, 9364.
Liu P., Young T. Z., Acar M.; Cell Rep. 2015, 13, 634.

1. A microfluidic unit for isolating and culturing yeast cells, comprising:
a medium inlet, a medium outlet, and a passage interconnecting said inlet and said outlet;
one single cell trapping chamber in the passage, said trapping chamber being a mother cell trapping chamber; characterized in that the microfluidic unit further comprises:
a daughter cells trapping chamber in the passage, downstream of the one single cell trapping chamber, and configured for retaining the daughter cells of a single cell trapped in the one single cell trapping chamber while allowing offsprings of said daughter cells to escape with a flow of the medium in said passage.

2. The microfluidic unit according to claim 1, wherein the daughter cells trapping chamber comprises a plurality of daughter cell sub-chambers and the passage comprises selective sub-passages each individually connecting one of said sub-chambers with the single cell trapping chamber.

3. The microfluidic unit according to claim 2, wherein the selective sub-passages connect with the single cell trapping chamber at distinct locations arranged side-by-side.

4. The microfluidic unit according to claim 2, wherein each of the selective sub-passages has a diameter or width greater than or equal to 2 μm and/or less than or equal to 5 μm.

5. The microfluidic unit according to claim 4, wherein the passage comprises exit sub-passages each individually connecting one of said sub-chambers downstream with the medium outlet, each of said sub-passages having a diameter greater than 5 μm.

6. The microfluidic unit according to claim 5, wherein the selective sub-passages extend parallel to each other along a main direction of the unit, the exit sub-passages extending at least partially transversely to said main direction.

7. The microfluidic unit according to claim 1, wherein the daughter cells trapping chamber forms a screen with openings, each opening being configured for retaining a daughter cell so that said chamber can trap a plurality of daughter cells.

8. The microfluidic unit according to claim 7, wherein the openings of the daughter cells trapping chamber are more than 80.

9. The microfluidic unit according to claim 7, wherein the openings of the daughter cells trapping chamber have, each, a diameter or width greater than or equal to tum and/or less than or equal to 5 μm.

10. The microfluidic unit according to claim 7, wherein the screen of the daughter cells trapping chamber extends along a main direction, the openings of said chamber being arranged in two parallel rows along said main direction.

11. The microfluidic unit according to claim 7, wherein the openings of the daughter cells trapping chamber are formed by pillars parallel to each other and extending between two substrates.

12. The imicrofluidic unit according to claim 7, wherein the single cell trapping chamber has an exit towards the daughter cells trapping chamber with a passage diameter or width greater than or equal to 2 μm and/or less than or equal to 5 μm.

13. The microfluidic unit according to claim 12, wherein the passage between the single cell trapping chamber and the daughter cells trapping chamber forms at least one meander.

14. The microfluidic unit according to claim 1, wherein said microfluidic unit further comprises:
a grand-mother cell trapping chamber in the passage, upstream of said mother cell trapping chamber.

15. The microfluidic unit according to claim 14, wherein the passage comprises a drain by-pass fluidly connecting the grand-mother cell trapping chamber to the medium outlet.

16. The microfluidic unit according to claim 14, wherein the drain by-pass has a diameter that is greater than 5 μm.

17. The microfluidic unit according to claim 14, wherein the passage between the grand-mother cell trapping chamber and the mother cell trapping chamber has a diameter or width that is greater than or equal to 2 μm and/or less than or equal to 5 μm.

18. A microfluidic device comprising:
a unique inoculation channel with a fluid inlet;
a unique waste channel with a fluid outlet; and
microfluidic units arranged side-by-side each with a medium inlet connected to the unique, inoculation channel and a medium outlet connected to the unique waste channel;
wherein each of the microfluidic unit is according to claim 1, and in that a set of inoculation channels are connected to form the unique inoculation channel, and in that a set of waste channels are connected to form the unique waste channel.

19. The microfluidic device according to claim 18, further comprising a rotatable basis supporting the unique inoculation channel, the unique waste channel and the microfluidic units, said units having each a main direction extending along a radius of said basis.

20. A platform comprising:
a liquid handling set up;
a computer;
a microscope with a camera; and
the microfluidic device according to claim 18.

21. The platform according to claim 20, wherein said platform further comprises a centrifuge.

22. A method of isolating and culturing a plurality of yeast cells comprising:
providing a platform for yeast aging analysis comprising a computer, a microscope with a camera, at least one microfluidic device according to claim 18, and a liquid handling set up or a centrifuge;
injecting suspended yeast cells through the unique inoculation channel;
trapping in each daughter cells trapping chambers, entire progeny of each mother cells captured in each single cell trapping chamber; and
taking photos of the daughter cells tapping chambers.
